(19) United States
(12) Patent Application Publication
(10) Pub. No.: US 2022/0175287 A1
(43) Pub. Date: Jun. 9, 2022

(54) METHOD AND DEVICE FOR DETECTING DRIVER DISTRACTION

(71) Applicant: SHENZHEN UNIVERSITY, Shenzhen (CN)

(72) Inventors: Guofa LI, Shenzhen (CN); Weiquan YAN, Shenzhen (CN); Weijian LAI, Shenzhen (CN); Yaoyu CHEN, Shenzhen (CN); Yifan YANG, Shenzhen (CN); Shenglong LI, Shenzhen (CN); Heng XIE, Shenzhen (CN); Xiaohang LI, Shenzhen (CN)

(21) Appl. No.: 16/629,944

(22) PCT Filed: Nov. 25, 2019
(86) PCT No.: PCT/CN2019/120566

§ 371 Date: Feb. 17, 2022

(30) Foreign Application Priority Data
Aug. 1, 2019 (CN) 201910707858.4

(51) Int. Cl. A61B 5/18; G06N 3/08; A61B 5/372; A61B 5/00; B60Q 9/00; G06K 9/62

(52) U.S. Cl. CPC A61B 5/18; G06N 3/08; A61B 5/372; G06K 9/6262; B60Q 9/00; G06K 9/6257; A61B 5/7267

(57) ABSTRACT

The present application is applicable to the field of computer application technology, and provides methods and devices for detecting driver distraction, including: acquiring the EEG data of the driver; preprocessing the EEG data, and then inputting it into a pre-trained distraction detection model to obtain the distraction detection result of the driver; obtaining the distracted detection model by training a preset convolution-recurrent neural network using EEG sample data and corresponding distracted result label; sending the distraction detection result to an in-vehicle terminal associated with the identity information of the driver, wherein the distraction detection result is used to trigger the in-vehicle terminal to generate driving reminder information according to the distraction detection result. When detecting driver distraction, the accuracy and efficiency are improved, thereby reducing the probability of traffic accidents.

The present application claims priority of the Chinese patent application No. CN201910707858.4, filed in the Chinese Patent Office on Aug. 1, 2019, with application No. 201910707858.4 and the title of invention “Method and Device for Detecting Driver Distraction”, the content of which is incorporated in the present application by reference.

### TECHNICAL FIELD

The present application relates to the field of computer application technology, and in particular to a method and devices for detecting driver distraction.

### BACKGROUND

Nowadays, the use rate of automobiles is increasing. Although the presence of automobiles has greatly facilitated the society, it has also brought huge traffic risks, especially traffic accidents. Since 2015, the rate of automobile accidents in China has increased significantly. This makes us have to ring the alarm. Wherein distracted driving occupies a very large part of traffic safety. According to actual road driving experiments by the National Highway Safety Administration, nearly 80% of collisions and 65% of critical collisions are related to distracted driving. And with the current popularity of in-vehicle entertainment equipment, mobile phones and other devices, the factors that lead to driving distraction are becoming more and more common.

In the prior art, Support Vector Machine (SVM) is used to detect whether the driver is distracted from driving. However, since the SVM uses quadratic programming to solve the support vector, while solving quadratic programming involves the calculation of matrix of order m, where m represents the number of samples, when m is very large, the storage and calculation of the matrix will consume a lot of machine memory and operating time. Therefore, in the prior art, when driver distraction detection is performed on a driver, there are problems of low detection efficiency and inaccuracy.

### SUMMARY

Embodiments of the present application provide a method and devices for detecting driver distraction, which can solve the problems of low detection efficiency and inaccuracy when performing distraction detection on a driver in the prior art.

In a first aspect, embodiments of the present application provide a method for detecting driver distraction, including:

acquiring electroencephalogram (EEG) data of a driver; preprocessing the EEG data, and inputting the EEG data into a distraction detection model that is pre-trained to obtain a distraction detection result of the driver, wherein the distracted detection model is obtained by training a preset recurrent neural network using EEG sample data and corresponding distraction result labels; and sending the distraction detection result to an in-vehicle terminal associated with identity information of the driver, wherein the distraction detection result is configured for triggering the in-vehicle terminal to generate driving reminder information according to the distraction detection result.

It should be understood that by detecting the EEG data of the driver acquired in real time according to the trained recurrent neural network to determine whether the driver is distracted, and performing a corresponding processing through a preset in-vehicle terminal when distraction is detected, the accuracy and efficiency of distraction detection of the driver are improved, therefore reducing the probability of traffic accidents.

In a second aspect, embodiments of the present application provide a device for detecting driver distraction, including a memory, a processor, and a computer program stored in the memory and executable on the processor. The processor implements the following steps when executing the computer program:

acquiring EEG data of a driver;

preprocessing the EEG data, and inputting the EEG data into a distraction detection model that is pre-trained to obtain a distraction detection result of the driver, wherein the distracted detection model is obtained by training a preset recurrent neural network using EEG sample data and corresponding distraction result labels;

sending the distraction detection result to an in-vehicle terminal associated with identity information of the driver, wherein the distraction detection result is configured for triggering the in-vehicle terminal to generate driving reminder information according to the distraction detection result.

In a third aspect, embodiments of the present application provide a device for detecting driver distraction, including:

an acquiring unit, configured for acquiring EEG data of a driver;

a detecting unit, configured for preprocessing the EEG data, and then inputting the EEG data to a distraction detection model that is pre-trained to obtain a distraction detection result of the driver, wherein the distraction detection model is obtained by training a preset recurrent neural network using EEG sample data and corresponding distraction result labels;

a sending unit, configured for sending the distraction detection result to an in-vehicle terminal associated with identity information of the driver, wherein the distraction detection result is configured for triggering the in-vehicle terminal to generate driving reminder information according to the distraction detection result.

In a fourth aspect, embodiments of the present application provide a computer readable storage medium. The computer storage medium stores a computer program, the computer program includes program instructions, and the program instructions, when executed by the processor, cause the processor to perform the method according to the first aspect.

In a fifth aspect, embodiments of the present application provide a computer program product that, when the computer program product is running on a terminal device, causes the terminal device to perform the method for detecting driver distraction according to any one of the first aspects.

Details of one or more embodiments of the present application are set forth in the drawings and description below. Other features, objects, and advantages of the present application will become apparent from the specification, drawings, and claims.

### BRIEF DESCRIPTION OF THE DRAWINGS

In order to more clearly illustrate the technical solutions in the embodiments of the present application, the drawings needed in the embodiments or in the description of the prior art will be briefly introduced below. Obviously, the drawings in the following description are only some embodiments of the present application, and other drawings may be obtained by those of ordinary skill in the art according to these drawings without paying creative labor.

FIG. 1 is a flowchart of a method for detecting driver distraction according to Embodiment 1 of the present application;

FIG. 2 is a flowchart of a method for detecting driver distraction according to Embodiment 2 of the present application;

FIG. 3 is a schematic diagram showing model training and detection application according to Embodiment 2 of the present application;

FIG. 4 is a schematic diagram showing the preprocessing flow of EEG data according to Embodiment 2 of the present application;

FIG. 5 is a diagram showing the positions of electrodes on an acquisition device according to Embodiment 2 of the present application;

FIG. 6 is a schematic diagram of artifact analysis according to Embodiment 2 of the present application;

FIG. 7 is a schematic diagram showing large noise and the selection and removal thereof in EEG according to Embodiment 2 of the present application;

FIG. 8 is a schematic diagram of a sequential driving distraction prediction convolution-recurrent neural network according to Embodiment 2 of the present application;

FIG. 9 is a schematic diagram showing a recurrent structure of a gated recurrent unit according to Embodiment 2 of the present application;

FIG. 10 shows graphs of detection results of three network structures according to Embodiment 2 of the present application;

FIG. 11 is a schematic diagram of a device for detecting driver distraction according to Embodiment 3 of the present application;

FIG. 12 is a schematic diagram of a device for detecting driver distraction according to Embodiment 4 of the present application.

### DETAILED DESCRIPTION

In the following description, for the sake of explanation rather than limitation, specific details such as specific system structure and technology are proposed so that the embodiments of the present application can be fully understood. However, it should be clear to those of ordinary skill in the art that the present application can also be implemented in other embodiments without these specific details. In other cases, detailed descriptions of well-known systems, devices, circuits, and methods are omitted so as to avoid unnecessary details hindering the description of the present application.

Referring to FIG. 1, FIG. 1 is a flowchart of a method for detecting driver distraction according to Embodiment 1 of the present application. The execution subject of the method for detecting driver distraction in this embodiment is a device with the function of detecting driver distraction, and the device includes, but is not limited to, a computer, a server, a tablet computer, or a terminal. The method for detecting driver distraction as shown in the figure may include the following steps:

S101: Acquiring EEG data of a driver.

Nowadays, the use rate of automobiles is increasing. Although the presence of automobiles has greatly facilitated the society, it has also brought huge traffic risks, especially traffic accidents. Since 2015, the rate of automobile accidents in China has increased significantly. This makes us have to ring the alarm. Where distracted driving occupies a very large part of traffic safety. According to actual road driving experiments by the National Highway Safety Administration, nearly 80% of collisions and 65% of critical collisions are related to distracted driving. Therefore, the detection of distracted driving is particularly important. And with the current popularity of in-vehicle entertainment equipment, mobile phones and other devices, the factors that lead to driving distraction are becoming more and more common. Therefore, it is necessary to detect the distraction state of the driver to improve road safety. As an automobile operator, the driving performance of the driver often has a great impact on local traffic conditions. Unsafe driving manner, fatigue driving, and distracted driving all pose great threats to road safety. Many researchers have studied the effects of distraction on road safety. If the driver's distracted state, fatigue state, etc. can be predicted in advance, the driver can be reminded in dangerous situations, thereby providing greater guarantee for road traffic safety, and providing theoretical basis for road traffic safety. Research on the prediction of driver's driving state has a positive effect on the safety of the traffic system, in addition to easing urban traffic pressure and effectively reducing the incidence of traffic accidents, it can also play a role in the handover right between automatic driving and manual driving in the future automobile auxiliary driving system.

In order to solve the problem of distracted driving, people have proposed many methods for detecting the current mental state of human. Research on the prediction of driver's driving state is mainly to improve driving safety and traffic safety. This embodiment mainly studies the method for predicting driving state. The preprocessed EEG signals of the driver are used as input features, and the driving state is identified through a convolutional neural network to predict the driving state information of the driver, so as to make early warning for dangerous driving behavior, thereby reducing the occurrence of traffic accidents, and improving driving safety. And at the same time, a new idea for the processing of EEG signals is provided, if there is enough database support, the processing of time-domain EEG signals should still have great potential.

S102: Preprocessing the EEG data, and inputting the EEG data into a distraction detection model that is pre-trained to obtain a distraction detection result of the driver, wherein the distracted detection model is obtained by training a preset recurrent neural network using EEG sample data and corresponding distraction result labels.

In traditional analysis, the EEG data are usually transformed from time domain to frequency domain for analysis. However, in this case, the time signal in the time domain is destroyed, even in some other improved methods, the information in the time domain is more or less destroyed, and the simplified information may not truly reflect the entire EEG. This embodiment hopes to use the powerful computing performance of the present computers to directly process EEG signals in the time domain through neural network. Although the recognition rate of the final test set is only 85%, which is equivalent to the traditional methods, neural network is often better at processing big data, as there are not many EEG data, only 18 hours, collected in this experiment, it is believed that the potential of using neural network to process EEG signals is huge by establishing huge database support.

In the whole research, firstly, a training module is carried out. Cleaned EEG data are acquired through sample collection and preprocessing, then the cleaned EEG data are used to train the convolution-recurrent neural network (CSRN) for detecting driver distraction in this embodiment, and network structure parameters are continuously adjusted to optimize the network structure, so as to obtain the best network parameters. The adjusted network model is applied to the vehicle system as an actual model, if there is an instrument for collecting EEG, the trained network can be used to predict the distracted state of the driver in real time, and the distracted state will be fed back to the auxiliary driving system to make a reasonable decision.

Before the development of convolutional neural networks, the commonly used network structure was a multi-layer perceptron. In theory, a multi-layer fully connected layer can also fit any polynomial function, but in fact, the effect is not good, because in order to fit a sufficiently complex function, the multi-layer perceptron needs a very large number of parameters to support, which not only increases the difficulty of training, but also easily falls into the phenomenon of over-fitting. In addition, if the input is an image, every pixel will be connected to each neuron in the next layer, which causes the network to be too sensitive to location and weak in generalization ability. Once the same target appears in different regions, the network needs to be retrained, and for images of different sizes, the input of the network is fixed, thus images of different sizes must be cropped and transformed into images of a specified size before input.

Due to many shortcomings of multi-layer perceptron, convolutional neural networks have emerged. Convolutional neural network is a type of feedforward neural network with convolutional calculation and deep structure, and it is one of the representative algorithms of deep learning. In each convolutional layer, there is a convolution kernel of a specified size, and the convolution kernel completes the convolution operation on the whole data according to a given step size. Therefore, it can be considered that the sensitivity of the network to the location is reduced, and the network is compatible with data of different sizes. Convolutional neural networks have been proven to be very effective in feature extraction by many experiments. Nowadays, many image recognition technologies are also based on convolutional neural networks. The network structure of the present application also uses convolutional layers, which has achieved good results.

In this embodiment, the recurrent neural network is used to train the sample data, and the recurrent neural network in this embodiment includes a convolution-recurrent structure. Specifically, the first three layers of networks is a convolutional unit. The data in each layer of networks reach the next layer after convolution, pooling, batch normalization, and activation. The output of the convolutional unit is used as the input of gated recurrent unit, and a feature vector of a preset length is obtained after the gated recurrent unit, such as a feature vector of 128 digital bits. The feature vector is input into the fully connected layer, so as to finally obtain an output which detects whether the driver is currently distracted.

Further, after step S102 the method may further include: sending the distraction detection result to an auxiliary driving device preset in a vehicle for assisting the driver to drive safely if the distraction detection result is that the driver is distracted.

In this embodiment, an auxiliary driving device is preset on the vehicle. The auxiliary driving device in this embodiment is used to assist the driver in driving, for example, when the driver is distracted, a corresponding reminder can be provided, or security protection can be provided, such as improving the level of security protection. When the current EEG data of the driver is detected by the recurrent neural network obtained through the above training and the distraction detection result of the driver is obtained, the distraction detection result will be sent to the auxiliary driving device to assist the driver to drive safely if the distraction detection result is that the driver is distracted.

S103: Sending the distraction detection result to an in-vehicle terminal associated with identity information of the driver, wherein the distraction detection result is configured for triggering the in-vehicle terminal to generate driving reminder information according to the distraction detection result.

In this embodiment, the vehicle is equipped with an in-vehicle terminal, which is used to trigger the in-vehicle terminal to generate driving reminder information according to the distraction detection result. Specifically, after the driver's distraction is detected, driving reminder information, such as a voice message, is generated to remind the driver to concentrate on driving, or music is played to relieve the driving fatigue of the driver, which is not limited here.

In the above solution, the EEG data of the driver are acquired; the EEG data are pre-processed, and then input into a pre-trained distraction detection model to obtain the distraction detection result of the driver, wherein the distracted detection model is obtained by training a preset recurrent neural network using EEG sample data and corresponding distraction result labels; and the distraction detection result is send to an in-vehicle terminal associated with the identity information of the driver, wherein the distraction detection result is used to trigger the in-vehicle terminal to generate driving reminder information according to the distraction detection result. The EEG data of the driver obtained in real time is detected by trained recurrent neural network, thereby judging whether the driver is distracted. And when the distraction is detected, the corresponding processing is performed through the preset in-vehicle terminal, which improves the accuracy and efficiency when detecting driver distraction, thereby reducing the probability of traffic accidents.

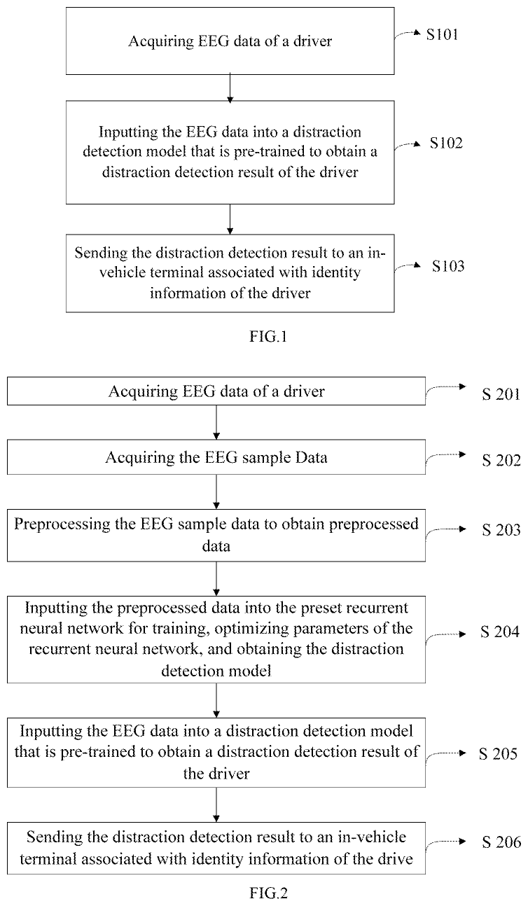

Referring to FIG. 2, FIG. 2 is a flowchart of a method for detecting driver distraction according to Embodiment 2 of the present application. The execution subject of the method for detecting driver distraction in this embodiment is a device with the function of detecting driver distraction, and the device includes, but is not limited to, a computer, a server, a tablet computer, or a terminal. The method for detecting driver distraction as shown in the figure may include the following steps:

S201: Acquiring EEG data of a driver.

The implementation manner of S201 in this embodiment is exactly the same as that of S101 in the embodiment corresponding to FIG. 1. For details, please refer to the related description of S101 in the embodiment corresponding to FIG. 1, which will not be repeated here.

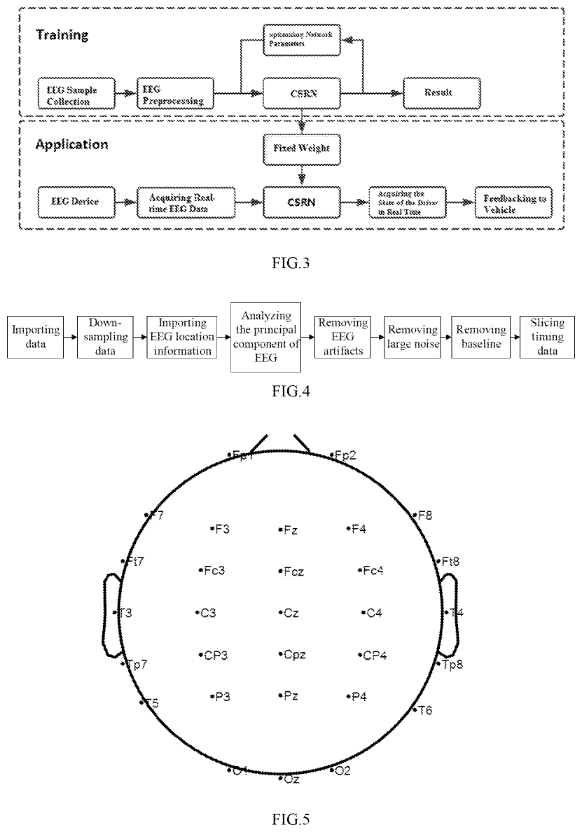

Also referring to FIG. 3, FIG. 3 is a schematic diagram showing model training and detection application according to this embodiment. During training, cleaned EEG data are acquired through EEG sample collection and EEG preprocessing, the cleaned EEG data are used to train CSRN network, and network structure parameters are continuously adjusted to optimize the network structure, so as to obtain the best network parameters, that is, to obtain a CSRN network with fixed parameter weights. The adjusted network model is applied to vehicle system as an actual model. The real-time EEG data can be acquired by an EEG device for collecting EEG. The trained CSRN network can be used to detect and obtain the driver's distracted state in real time, and finally the distracted state is fed back to the vehicle, such as to a preset auxiliary driving device in the vehicle, so as to make a reasonable decision and regulation.

S202: Acquiring the EEG sample data.

This embodiment hopes to use the powerful computing performance of the present computers to directly process EEG signals in the time domain through neural network. Although the recognition rate of the final test set is only 85%, which is equivalent to the traditional methods, neural network is often better at processing big data. In this embodiment, a huge database support is established by collecting EEG data. In the actual test process, the experimenter collected 18-hours data from the test subject and established a huge database support. It is believed that the potential of using neural network to process EEG signals is huge.

S203: Preprocessing the EEG sample data to obtain preprocessed data.

In the whole research, firstly, a training module is carried out. Cleaned EEG data are acquired through sample collection and preprocessing, then the cleaned EEG data are used to train CSRN network, and network structure parameters are continuously adjusted to optimize the network structure, so as to obtain the best network parameters. The adjusted network model is applied to the vehicle system as an actual model, if there is an instrument for collecting EEG, the trained network can be used to predict the distracted state of the driver in real time, and the distracted state will be fed back to the auxiliary driving system to make a reasonable decision.

Also referring to FIG. 4, FIG. 4 is a schematic diagram showing the preprocessing flow of EEG data. EEG signals are very weak, such that an amplifier with extremely high amplification is needed to capture the EEG signals. In practice, EEG often has a lower signal-to-noise ratio. In addition to high frequency noise and industrial frequency (50 Hz) noise, clutter with frequencies similar to those of EEG will also be mixed into the EEG signals. The clutter in EEG signals is often referred to as artifact, and the artifacts in this embodiment may include ocular artifact, myoelectric artifact, electrocardiographic artifact, and the like. The EEG signal without removing artifacts has a very low signal-to-noise ratio and cannot be used directly, therefore, it is necessary to go through a preprocessing step. In the main process of EEG preprocessing, the preprocessed data are obtained by importing data, down-sampling data, importing EEG location information, analyzing the principal component of EEG, removing EEG artifacts, removing large noise, removing baseline and finally slicing timing data.

Further, step S203 includes:

S2031: Acquiring identification information of collection points corresponding to the EEG sample data, and determining first position information of electrodes corresponding to the identification information of the collection points on a data acquisition device.

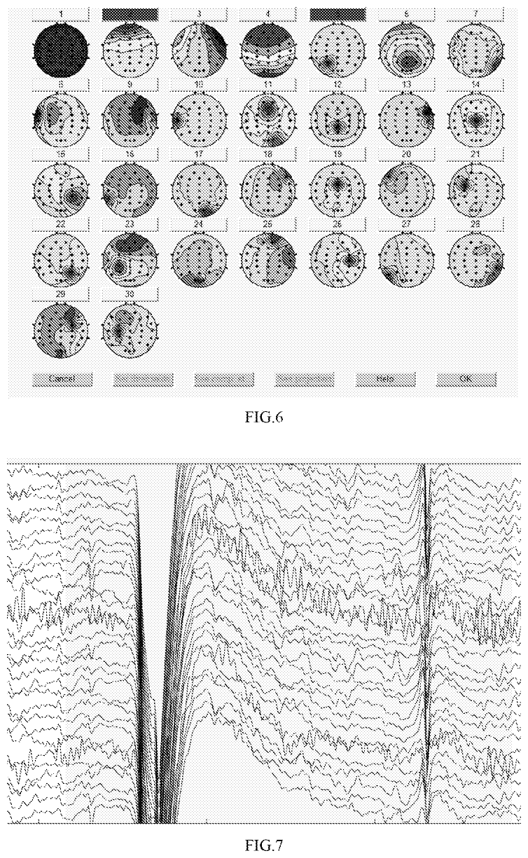

Also referring to FIG. 5, FIG. 5 is a diagram showing the position of electrodes on an acquisition device used in this embodiment, where, all the marks in the figures, such as C3˜C5, Cp3˜Cp5, F3˜F4, Fc1˜Fc2, Fp1˜Fp2, O1˜O2, P3˜P4, T4˜T5 and Tp7˜Tp8, are used to indicate corresponding electrode marks at different acquisition positions on the acquisition device. The acquisition device in this embodiment may be an EEG cap. Because the number and position of the electrodes of different types of EEG caps are different, for EEG data, it is necessary to input the electrode position information of EEG, so as to perform the principal component analysis of EEG.

Further, before step S2031, the method further includes: performing frequency reduction processing on the EEG sample data; and enabling frequency-reduced EEG sample data to pass through a low-pass filter with a preset frequency to obtain filtered EEG sample data.

Specifically, the sampling frequency of most EEG devices is very high. Here we reduce the frequency of EEG data to 100 Hz so as to reduce calculation amount. In addition, the data pass through a low-pass filter with a preset frequency, such as a low-pass filter with an upper cut-off frequency of 50 Hz, to remove irrelevant high frequency noise and industrial frequency noise.

S2032: Determining, according to the first position information, second position information of emission sources on cerebral cortex corresponding to the collection points.

Since the electrodes of the EEG cap are artificially determined, the EEG cap only represents the receiving source of EEG, and does not represent the emission source of EEG. There is a superposition effect of multiple emission sources on each sampling electrode of EEG, therefore, it is necessary to relocate the emission source of EEG signal (i.e., the second position information) through the EEG position information (i.e., the first position information).

It should be noted that, in this embodiment, in order to easily distinguish and reflect the difference and connection between the electrode position and the position of emission source of cerebral cortex, the position information of the collection electrode on the data acquisition device is represented by the first position information, and the position information corresponding to the emission source of EEG is represented by the second position information.

Further, step S2032 includes: determining electrodes corresponding to the first position information on the data acquisition device; determining second position information of emission sources corresponding to the electrodes, wherein the emission sources are regions on the cerebral cortex where EEG sample data are generated.

Specifically, in this embodiment, after the first position information is determined, the electrodes corresponding to the first position information on the data acquisition device are determined according to the first position information, and then the second position information of the emission sources corresponding to the electrodes is determined. The emission sources in this embodiment are used to indicate regions on the cerebral cortex where EEG sample data are generated.

S2033: Removing artifacts in the EEG sample data according to the second position information, and slicing according to a preset slice period to obtain the preprocessed data, wherein the artifacts are EEG sample data corresponding to set positions to be removed.

Since the electrodes of the EEG cap are artificially determined, the EEG cap only represents the receiving means of EEG, and does not represent the emission source of EEG. There is a superposition effect of multiple emission sources on each sampling electrode of EEG, therefore, it is necessary to relocate the emission sources of EEG signal through the EEG position information. In addition, independent component analysis can also locate the emission sources of some artifacts so as to remove artifacts.

The working principle of independent component analysis is as follows: in this embodiment, it can be assumed that n emission sources in the brain are transmitting EEG signals at the same time, and the experiment uses an EEG cap with n electrodes to collect the signals from the n emission sources, after a period of time, a set of data can be obtained.

x∈{x⁽ⁱ⁾;i=1, . . . ,m}, where, in represents the number of samples.

Assuming that the n emission sources of EEG are:

s={s¹, s², . . . , sⁿ}^{T}, s∈Rⁿ, where, each of these dimensions is an independent source, let A be an unknown mixed matrix used to superimpose EEG signals, that is:

x=[x⁽¹⁾,x⁽²⁾, . . . ,x^{(m)}]=[As⁽¹⁾,As⁽²⁾, . . . ,As^{(m)}]=As

Since both A and s are unknown, s needs to be derived from x, and this process is also referred as blind source separation. Assuming W=A⁻¹, then s⁽ⁱ⁾=Wx⁽ⁱ⁾. Assuming there is a random variable S with probability density function pₛ (s), where continuous values are probability density functions, and discrete values are probabilities. For simplicity, let's assume that s represents real number and there is a random variable x=As, where, A and x are both real numbers. Let pₓ(x) be the probability density of x Assuming that the probability density function is p(x) and its corresponding cumulative distribution function is F(x), where the derivation formula for pₓ(x) is:

Fₓ(x)=P(X≤x)=P(As≤x)=P(s≤Wx)=Fₛ(Wx)

pₓ(x)=Fₓ′(x)=Fₛ′(Wx)=p(Wx)|W|

Then, the maximum likelihood estimation can be used to calculate the parameter W, assuming that each sᵢ has a probability density pₛ, then the joint distribution of the signal source at a given moment is:

$$p ( s ) = {\prod }_{i = 1}^{n} {p}_{s} ( {s}_{i} )$$

This formula assumes that the signal from each signal source is independent. It can be obtained from the derivation formula of pₓ(x) that:

$$p ( x ) = {p}_{s} ( Wx ) \vert W \vert = \vert W \vert {\prod }_{i = 1}^{n} {p}_{x} ( {W}^{T} x )$$

Without prior knowledge, W and s cannot be obtained, therefore, it is necessary to know pₛ (s). Let's pick a probability density function and assign it to s. Since the probability density function p (x) is derived from the cumulative distribution function F(x), and conventional F(x) needs to meet two properties, that is, the function is monotonically increasing and its range is [0,1], and the threshold function sigmoid function meets this condition. Therefore, it is assumed that the cumulative distribution function of s conforms to the sigmoid function:

$$g ( s ) = \frac{1}{1 + {e}^{- s}}$$

After the differentiation, then:

$${p}_{s} ( s ) = {g}^{′} ( s ) = \frac{{e}^{s}}{{( 1 + {e}^{s} )}^{2}} ;$$

After knowing this, only W needs to be confirmed, so, with a given EEG collector x, the log-likelihood estimation can be derived:

$$l ( W ) = log {\prod }_{i = 1}^{m} p ( {x}^{( i )} ) = {\sum }_{i = 1}^{m} ( {\sum }_{j = 1}^{n} log {g}^{′} ( {W}_{i}^{T} {x}^{( i )} ) + \vert W \vert )$$

Next, we can differentiate and iterate over W, and only learning rate α need to be specified to get W:

$$W := W + a ( [ \begin{matrix}1 - 2 g ( {W}_{1}^{T} {x}^{( i )} ) \\ 1 - 2 g ( {W}_{2}^{T} {x}^{( i )} ) \\ … \\ 1 - 2 g ( {W}_{n}^{T} {x}^{( i )} )\end{matrix} ] {x}^{{( i )}^{T}} + {( {W}^{T} )}^{- 1} )$$

In this experiment, after completing the independent component analysis, 30 calculated emission sources of EEG can be obtained for the next step of artifact removal.

Also referring to FIG. 6, FIG. 6 is a schematic diagram of artifact analysis according to this embodiment. After performing the principal component analysis calculation, the calculated 30 new emission sources can be obtained, even if there is a gap with the real sources. Here the artifact removal plug-in of the Matrix Laboratory (MATLAB) can be used to remove artifacts. The 30 relocated sources are shown in FIG. 6, and the sources to be removed can be directly selected and removed.

Also referring to FIG. 7, FIG. 7 is a schematic diagram showing large noise and the selection and removal thereof in EEG according to this embodiment. In practical applications, there are often unavoidable situations that lead to the subjects shaking a lot or the electrodes falling. When this happens, EEG often has a huge waveform jitter which needs to be manually removed. EEG processing plug-in in MATLAB can be used to select unwanted waveforms and remove them directly.

EEG data reflect a dynamic change in brain potential. DC signals cannot reflect the information of the brain, therefore, in the analysis of EEG signals, DC components need to be removed. In addition, the phenomenon of baseline drift also occurs in the step of removing large noise, therefore, removing DC component is completed in the final step of EEG preprocessing. The current DC component can be obtained by calculating the average value of the data of each channel of EEG, and the DC component is removed by subtracting this component from the data.

EEG signal in the time domain is too long to be directly input to the neural network for training. Here we slice EEG data into short-term time series, i.e., slicing according to a preset slice cycle within a preset period, for example, the data of 15 minutes is divided into data of 2 seconds, so as to reduce the calculation amount of the neural network and improve the real-time performance of the network. Each slice is marked as the corresponding state, including the state of distracted driving and normal driving.

S204: Inputting the preprocessed data into the preset recurrent neural network for training, optimizing parameters of the recurrent neural network, and obtaining the distraction detection model.

Before the development of convolutional neural networks, the commonly used network structure was a multi-layer perceptron. In theory, a multi-layer fully connected layer can also fit any polynomial function, but in fact, the effect is not good, because in order to fit a sufficiently complex function, the multi-layer perceptron needs a very large number of parameters to support, which not only increases the difficulty of training, but also easily falls into the phenomenon of over-fitting. In addition, if the input is an image, every pixel will be connected to each neuron in the next layer, which causes the network to be too sensitive to location and weak in generalization ability. Once the same target appears in different regions, the network needs to be retrained, and for images of different sizes, the input of the network is fixed, thus images of different sizes must be cropped and transformed into images of a specified size before input. Due to many shortcomings of multi-layer perceptron, convolutional neural networks have emerged. Convolutional neural network is a type of feedforward neural network with convolutional calculation and deep structure, and it is one of the representative algorithms of deep learning. In each convolutional layer, there is a convolution kernel of a specified size, and the convolution kernel completes the convolution operation on the whole data according to a given step size. Therefore, it can be considered that the sensitivity of the network to the location is reduced, and the network is compatible with data of different sizes.

Convolutional neural networks have been proven to be very effective in feature extraction by many experiments. Nowadays, many image recognition technologies are also based on convolutional neural networks. The network structure of the present application also uses convolutional layers, which has achieved good results.

EEG signals are relatively special. They have spatial information at the same time point, and EEG signals sent from different positions of the brain also have temporal information, i.e., time domain signals. Therefore, this embodiment combines the advantages of convolutional neural network and recurrent neural network. In the first few layers of the network, the convolutional neural unit is used to extract the spatial characteristics of single time point, and then the processed data are input into a gated recurrent unit that is sensitive to time series so as to find the time characteristics of the data, and a final obtained feature vector of 128-bits length is input to the state classification network of the full connected layer.

Also referring to FIG. 8, FIG. 8 is a schematic diagram of a sequential driving distraction prediction recurrent neural network according to this embodiment. In the figure, the first three layers of networks is a convolutional unit, and the data in each layer of the network reaches the next layer after convolution, pooling, batch normalization, and activation. Specifically, firstly, preprocessed b×200×30 data are input, and b×5×6×200×1 data are obtained through the first layer of 3×3×3 convolution kernel and the first layer of 1×2×2 pooling window; then b×2×3×100×64 data are obtained by inputting the b×5×6×200×1 data to the second layer of 3×3×3 convolution kernel and the second layer of 1×1×2 pooling window; then b×1×1×25×512 data are obtained by inputting the b×2×3×100×64 data to the third layer of 2×1×3 convolution kernel and the second layer of 1×1×2 pooling window. Further, the recurrent neural network in this embodiment includes a gated recurrent unit, and the output of the convolutional unit is used as an input of the gated recurrent unit. The gated recurrent node in the gated recurrent unit in this embodiment is set as 512 bits of input data, 128 bits of hidden layer and 4 layers. After going through the gated recurrent unit, a feature vector of 128-bits length is obtained, which is input to the fully connected layers so as to obtain the final output. The three fully connected layers are b×128, b×64 and b×16 respectively, the final output data are b×2, and finally the detection result that whether the driver is in a distracted state is obtained.

Further, step S204 includes: inputting the preprocessed data into the recurrent neural network for convolution to obtain a convolution result, inputting the convolution result into a preset gated recurrent unit to obtain a feature vector, and inputting the feature vector to preset fully connected layers to obtain a detection result; and optimizing the parameters of the recurrent neural network according to difference between the detection result and its corresponding distraction result label so as to obtain the distraction detection model, wherein the gated recurrent unit is used to control data flow direction and data flow amount in the convolution-recurrent neural network.

Specifically, when processing time signals or other signal series, it is easy to find the deficiencies of traditional neural networks and convolutional neural networks. In a series, such as an article, it is very likely that a previous word is related to a next word, even a previous paragraph is related to a next paragraph, and traditional neural networks cannot build such a connection. Although a convolutional neural network can build connections between adjacent regions and capture features, once it exceeds beyond the range of the convolution kernel, it is impossible to extract such features, which is a fatal disadvantage in long series, and the recurrent neural network solves this problem well.

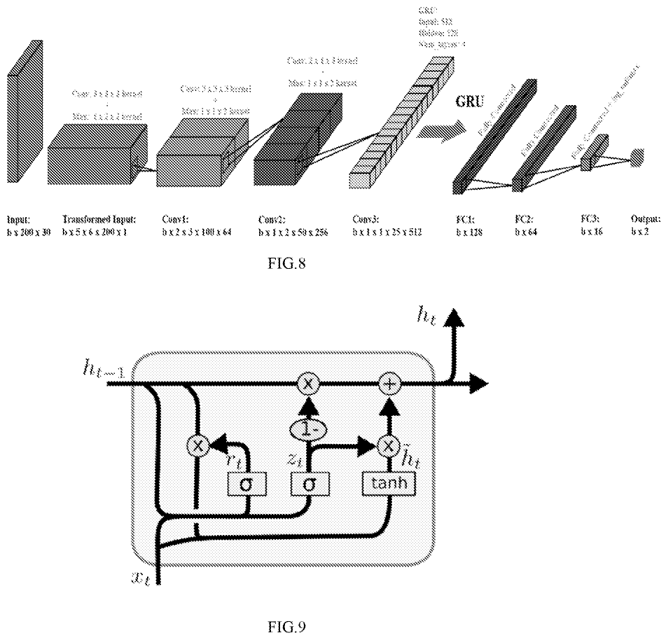

Also referring to FIG. 9, FIG. 9 is a schematic diagram showing a recurrent structure of a gated recurrent unit according to this embodiment. Where, the gated recurrent unit xₜ represents the input x at the current moment, and hₜ₋₁ represents the output at the previous moment. There are two gates in each recurrent unit, namely update gate zₜ and reset gate rₜ. The update gate is used to control the degree to which the state information of the previous moment is brought into the current state. The larger the value of the update gate is, the more state information of the previous moment is brought into the current state. The reset gate is used to control the degree to which the state information of the previous moment is ignored. The smaller the value of the reset gate is, the more state information of the previous moment is ignored. Compared with the traditional neural networks, this structure can better pass the information of the previous series to the back, because when the traditional recurrent neural network is trained to a deep level, the previous information has been ignored, and the gated recurrent unit can control retained information and ignored information, so it performs better in recurrent neural networks.

S205: Preprocessing the EEG data, and inputting the EEG data into a distraction detection model that is pre-trained to obtain a distraction detection result of the driver, wherein the distracted detection model is obtained by training a preset recurrent neural network using EEG sample data and corresponding distraction result labels;

In this embodiment, the recurrent neural network is used to train the sample data, and the recurrent neural network in this embodiment includes a convolution-recurrent structure. Specifically, the first three layers of networks is a convolutional unit. The data in each layer of networks reach the next layer after convolution, pooling, batch normalization, and activation. The output of convolutional network is used as the input of gated recurrent unit, and a feature vector of 128-bits length is obtained after going through the gated recurrent unit. The feature vector is input into the fully connected layer, so as to finally obtain an output which detects whether the driver is currently distracted.

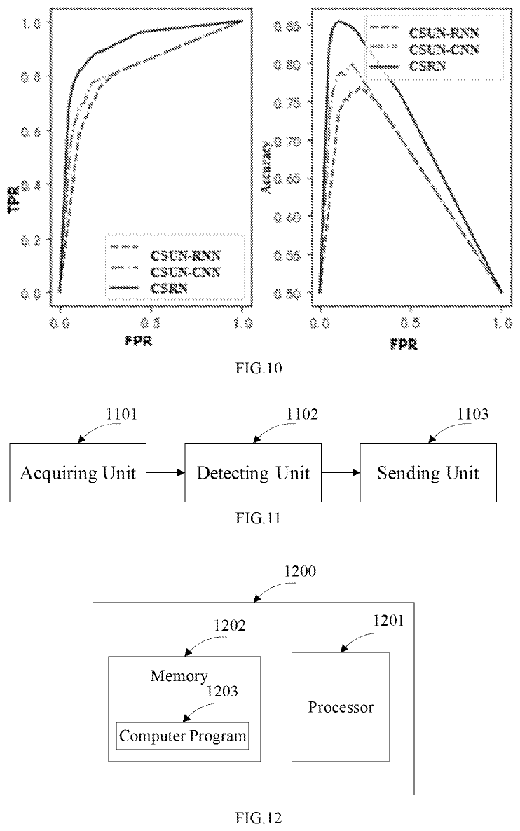

Also referring to FIG. 10 and Table 2, FIG. 10 shows graphs of detection results of three network structures according to this embodiment. Table 2 includes the identification performance of each of the three networks. Where the true positive rate represents the proportion of positive examples being correctly identified, and the false positive rate represents the proportion of negative examples being incorrectly identified as positive examples. In this embodiment, three network structures are compared, including our final convolution-recurrent network, and convolutional neural network and recurrent neural network. All the three networks are 7-layer networks, where the convolutional neural network does not add a recurrent unit and is not sensitive to time series, while recurrent neural network does not add convolution nodes and is not sensitive to the spatial location distribution of EEG. The convolution-circulation model combines characteristics of convolution model and recurrent model, so it performs best, reaching a recognition accuracy of 85%, while the convolution model and the recurrent model only have a recognition accuracy of 78% and 76% respectively.

S206: Sending the distraction detection result to an in-vehicle terminal associated with identity information of the driver, wherein the distraction detection result is configured for triggering the in-vehicle terminal to generate driving reminder information according to the distraction detection result.

In this embodiment, the vehicle is equipped with an in-vehicle terminal, which is configured to trigger the in-vehicle terminal to generate driving reminder information according to the distracted detection result. Specifically, after the driver's distraction is detected, driving reminder information, such as a voice message, is generated to remind the driver to concentrate on driving, or music is played to relieve the driving fatigue of the driver, which is not limited here.

Referring to FIG. 11, FIG. 11 is a schematic diagram of a device for detecting driver distraction according to Embodiment 3 of the present application. The device 1100 for detecting driver distraction may be a mobile terminal such as a smart phone or a tablet computer. Units included in the device 1100 for detecting driver distraction in this embodiment are used for performing steps in the embodiment corresponding to FIG. 1. For details, please refer to FIG. 1 and related descriptions in the embodiment corresponding to FIG. 1, which will not be repeated here. The device 1100 for detecting driver distraction in this embodiment includes:

an acquiring unit 1101, configured for acquiring EEG data of a driver;

a detecting unit 1102, configured for preprocessing the EEG data, and then inputting the EEG data to a distraction detection model that is pre-trained to obtain a distraction detection result of the driver, wherein the distraction detection model is obtained by training a preset recurrent neural network using EEG sample data and corresponding distraction result labels;

a sending unit 1103, configured for sending the distraction detection result to an in-vehicle terminal associated with identity information of the driver, wherein the distraction detection result is configured for triggering the in-vehicle terminal to generate driving reminder information according to the distraction detection result.

Referring to FIG. 12, FIG. 12 is a schematic diagram of a device for detecting driver distraction according to Embodiment 4 of the present application. The device 1200 for detecting driver distraction in this embodiment as shown in FIG. 12 may include: a processor 1201, a memory 1202, and a computer program 1203 stored in the memory 1202 and executable on the processor 1201. The steps in the foregoing method embodiments for detecting driver distraction are implemented when the processor 1201 executes the computer program 1203. The memory 1202 is used to store a computer program, and the computer program includes program instructions. The processor 1201 is used to execute program instructions stored in the memory 1202. Where, the processor 1201 is configured for calling the program instructions to perform the following operations:

The processor 1201 is configured for:
acquiring EEG data of a driver;
preprocessing the EEG data, and inputting the EEG data into a distraction detection model that is pre-trained to obtain a distraction detection result of the driver, wherein the distracted detection model is obtained by training a preset recurrent neural network using EEG sample data and corresponding distraction result labels;
sending the distraction detection result to an in-vehicle terminal associated with identity information of the driver, wherein the distraction detection result is configured for triggering the in-vehicle terminal to generate driving reminder information according to the distraction detection result.

It should be understood that, in the embodiments of the present application, the processor 1201 may be a Central Processing Unit (CPU), and the processor may also be another general purpose processor or a Digital Signal Processor (DSP), an Application Specific Integrated Circuit (ASIC), a Field-Programmable Gate Array (FPGA), or other programmable logic devices, discrete gate or transistor logic devices, discrete hardware components, etc. A general purpose processor may be a microprocessor or any conventional processor, or the like.

The memory 1202 may include a read-only memory and a random access memory, and provides instructions and data to the processor 1201. A part of the memory 1202 may further include a non-volatile random access memory. For example, the memory 1202 may also store information of device types.

In specific implementation, the processor 1201, the memory 1202, and the computer program 1203 described in the embodiments of the present application can execute the implementation manner described in the embodiment 1 and embodiment 2 of the method for detecting driver distraction provided in the embodiments of the present application, and can also execute the implementation manner of the terminal described in the embodiments of the present application, details are not described herein again.

In another embodiment of the present application, a computer-readable storage medium is provided. The computer-readable storage medium stores a computer program, where the computer program includes program instructions, and the program instructions are implemented when executed by a processor:

acquiring EEG data of a driver;

preprocessing the EEG data, and inputting the EEG data into a distraction detection model that is pre-trained to obtain a distraction detection result of the driver, wherein the distracted detection model is obtained by training a preset recurrent neural network using EEG sample data and corresponding distraction result labels;

sending the distraction detection result to an in-vehicle terminal associated with identity information of the driver, wherein the distraction detection result is configured for triggering the in-vehicle terminal to generate driving reminder information according to the distraction detection result.

The computer-readable storage medium may be an internal storage unit of the terminal according to any of the foregoing embodiments, such as a hard disk or a memory of the terminal. The computer-readable storage medium may also be an external storage device of the terminal, such as a plug-in hard disk, a Smart Media Card (SMC), a Secure Digital (SD) card, Flash Card, etc. Further, the computer-readable storage medium may include both an internal storage unit and an external storage device of the terminal. The computer-readable storage medium is used to store the computer program and other programs and data required by the terminal. The computer-readable storage medium may also be used to temporarily store data that has been or will be output.

In addition, each functional unit in embodiments of the present application may be integrated into one processing unit, or each of the units may exist separately physically, or two or more units may be integrated into one unit. The above integrated unit may be implemented in the form of hardware or in the form of software functional unit.

If the integrated unit is implemented in the form of a software functional unit and sold or used as an independent product, it may be stored in a computer-readable storage medium. Based on this understanding, the technical solution of the present application, in essence, or the part contributing to the prior art, or all or part of the technical solution can be embodied in the form of a software product. The software product is stored in a storage medium and includes a number of instructions for enabling a computer device (which may be a personal computer, a server, or a network device, etc.) to perform all or part of the steps of the method described in the embodiments of the present application. The foregoing storage media include: U disk, mobile hard disk, Read-Only Memory (ROM), Random Access Memory (RAM), magnetic disk or optical disk, and other media that can store program code. The above is only the specific implementation of the present application, but the scope of protection of the present application is not limited to this. Those of ordinary skill in the art can easily conceive of various equivalent modifications or substitutions within the technical scope of the present application. The modifications or substitutions should be covered by the protection scope of the present application. Therefore, the protection scope of the present application shall be subject to the protection scope of the claims.

1. A method for detecting driver distraction, comprising:
acquiring electroencephalogram (EEG) data of a driver;
preprocessing the EEG data, and inputting the EEG data into a distraction detection model that is pre-trained to obtain a distraction detection result of the driver, wherein the distracted detection model is obtained by training a preset recurrent neural network using EEG sample data and corresponding distraction result labels; and
sending the distraction detection result to an in-vehicle terminal associated with identity information of the driver, wherein the distraction detection result is configured for triggering the in-vehicle terminal to generate driving reminder information according to the distraction detection result.

2. The method for detecting driver distraction according to claim 1, characterized in that, before said inputting the EEG data into a distraction detection model that is pre-trained to obtain a distraction detection result of the driver, the method further comprises:
acquiring the EEG sample data;
preprocessing the EEG sample data to obtain preprocessed data; and
inputting the preprocessed data into the preset recurrent neural network for training, optimizing parameters of the recurrent neural network and obtaining the distraction detection model.

3. The method for detecting driver distraction according to claim 2, characterized in that, said inputting the preprocessed data into the preset recurrent neural network for training, optimizing parameters of the recurrent neural network and obtaining the distraction detection model comprises:
inputting the preprocessed data into the recurrent neural network for convolution to obtain a convolution result, inputting the convolution result into a preset gated recurrent unit to obtain a feature vector, and inputting the feature vector to preset fully connected layers to obtain a detection result; and optimizing the parameters of the recurrent neural network according to difference between the detection result and its corresponding distraction result label so as to obtain the distraction detection model, wherein the gated recurrent unit is used to control a data flow direction and a data flow amount in the recurrent neural network.

4. The method for detecting driver distraction according to claim 2, characterized in that, said preprocessing the EEG sample data to obtain preprocessed data comprises:
acquiring identification information of collection points corresponding to the EEG sample data, and determining first position information of electrodes corresponding to the identification information of the collection points on a data acquisition device;
determining, according to the first position information, second position information of emission sources on cerebral cortex corresponding to the collection points; and
removing artifacts in the EEG sample data according to the second position information, and slicing according to a preset slice period to obtain the preprocessed data, wherein the artifacts are EEG sample data corresponding to set positions to be removed.

5. The method for detecting driver distraction according to claim 4, characterized in that, before said acquiring identification information of collection points corresponding to the EEG sample data, and determining first position information of electrodes corresponding to the identification information of the collection points on a data acquisition device, the method further comprises:
performing frequency reduction processing on the EEG sample data; and
enabling frequency-reduced EEG sample data to pass through a low-pass filter with a preset frequency to obtain filtered EEG sample data.

6. The method for detecting driver distraction according to claim 1, characterized in that, after said inputting the EEG data into a distraction detection model that is pre-trained to obtain a distraction detection result of the driver, the method further comprises:
sending the distraction detection result to an auxiliary driving device preset in a vehicle for assisting the driver to drive safely if the distraction detection result is that the driver is distracted.

7. The method for detecting driver distraction according to claim 2, characterized in that, after said inputting the EEG data into a distraction detection model that is pre-trained to obtain a distraction detection result of the driver, the method further comprises:
sending the distraction detection result to an auxiliary driving device preset in a vehicle for assisting the driver to drive safely if the distraction detection result is that the driver is distracted.

8. The method for detecting driver distraction according to claim 3, characterized in that, after said inputting the EEG data into a distraction detection model that is pre-trained to obtain a distraction detection result of the driver, the method further comprises:
sending the distraction detection result to an auxiliary driving device preset in a vehicle for assisting the driver to drive safely if the distraction detection result is that the driver is distracted.

9. The method for detecting driver distraction according to claim 4, characterized in that, after said inputting the EEG data into a distraction detection model that is pre-trained to obtain a distraction detection result of the driver, the method further comprises:
sending the distraction detection result to an auxiliary driving device preset in a vehicle for assisting the driver to drive safely if the distraction detection result is that the driver is distracted.

10. The method for detecting driver distraction according to claim 5, characterized in that; after said inputting the EEG data into a distraction detection model that is pre-trained to obtain a distraction detection result of the driver, the method further comprises:
sending the distraction detection result to an auxiliary driving device preset in a vehicle for assisting the driver to drive safely if the distraction detection result is that the driver is distracted.

11. A device for detecting driver distraction, comprising:
an acquiring unit, configured for acquiring EEG data of a driver;
a detecting unit, configured for preprocessing the EEG data, and then inputting the EEG data to a distraction detection model that is pre-trained to obtain a distraction detection result of the driver, wherein the distraction detection model is obtained by training a preset recurrent neural network using EEG sample data and corresponding distraction result labels; and
a sending unit, configured for sending the distraction detection result to an in-vehicle terminal associated with identity information of the driver, wherein the distraction detection result is configured for triggering the in-vehicle terminal to generate driving reminder information according to the distraction detection result.

12. The device for detecting driver distraction according to claim 11, characterized in that, the device for detecting driver distraction further comprises:
a sample acquiring unit, configured for acquiring the EEG sample data of the driver;
a preprocessing unit, configured for preprocessing the EEG sample data to obtain preprocessed data; and
a training unit, configured for inputting the preprocessed data into the preset recurrent neural network for training, optimizing parameters of the recurrent neural network, and obtaining the distraction detection model.

13. A device for detecting driver distraction, comprising a memory, a processor, and a computer program stored in the memory and executable on the processor, characterized in that, the processor implements the following steps when executing the computer program:
acquiring EEG data of a driver;
preprocessing the EEG data, and inputting the EEG data into a distraction detection model that is pre-trained to obtain a distraction detection result of the driver, wherein the distracted detection model is obtained by training a preset recurrent neural network using EEG sample data and corresponding distraction result labels; and
sending the distraction detection result to an in-vehicle terminal associated with identity information of the driver, wherein the distraction detection result is configured for triggering the in-vehicle terminal to generate driving reminder information according to the distraction detection result.

14. The device for detecting driver distraction according to claim 13, characterized in that, before said inputting the EEG data into a distraction detection model that is pre-trained to obtain a distraction detection result of the driver, the device for detecting driver distraction further comprises:
acquiring the EEG sample data;
preprocessing the EEG sample data to obtain preprocessed data; and
inputting the preprocessed data into the preset recurrent neural network for training, optimizing parameters of the recurrent neural network and obtaining the distraction detection model.

15. The device for detecting driver distraction according to claim 14, characterized in that, said inputting the preprocessed data into the preset recurrent neural network for training, optimizing parameters of the recurrent neural network and obtaining the distraction detection model comprises:
inputting the preprocessed data into the recurrent neural network for convolution to obtain a convolution result, inputting the convolution result into a preset gated recurrent unit to obtain a feature vector, and inputting the feature vector to preset fully connected layers to obtain a detection result; and optimizing the parameters of the recurrent neural network according to difference between the detection result and its corresponding distraction result label so as to obtain the distraction detection model, wherein the gated recurrent unit is used to control data flow direction and data flow amount in the recurrent neural network.

16. The device for detecting driver distraction according to claim 14, characterized in that, said preprocessing the EEG sample data to obtain preprocessed data comprises:
acquiring identification information of collection points corresponding to the EEG sample data, and determining first position information of electrodes corresponding to the identification information of the collection points on a data acquisition device;
determining, according to the first position information, second position information of emission sources on cerebral cortex corresponding to the collection points; and
removing artifacts in the EEG sample data according to the second position information, and slicing according to a preset slice period to obtain the preprocessed data, wherein the artifacts are EEG sample data corresponding to set positions to be removed.

17. A computer-readable storage medium, the computer-readable storage medium stores a computer program, characterized in that, when the computer program is executed by a processor, the following steps are implemented:
acquiring EEG data of a driver;
preprocessing the EEG data, and inputting the EEG data into a distraction detection model that is pre-trained to obtain a distraction detection result of the driver; wherein the distracted detection model is obtained by training a preset recurrent neural network using EEG sample data and corresponding distraction result labels; and
sending the distraction detection result to an in-vehicle terminal associated with identity information of the driver, wherein the distraction detection result is configured for triggering the in-vehicle terminal to generate driving reminder information according to the distraction detection result.

18. The computer-readable storage medium according to claim 17, characterized in that, before said inputting the EEG data into a distraction detection model that is pre-trained to obtain a distraction detection result of the driver, further comprising:
acquiring the EEG sample data;
preprocessing the EEG sample data to obtain preprocessed data; and
inputting the preprocessed data into the preset recurrent neural network for training, optimizing parameters of the recurrent neural network, and obtaining the distraction detection model.

19. The computer-readable storage medium of claim 18, characterized in that, said inputting the preprocessed data into the preset recurrent neural network for training, optimizing parameters of the recurrent neural network, and obtaining the distraction detection model comprises:
inputting the preprocessed data into the recurrent neural network for convolution to obtain a convolution result, inputting the convolution result into a preset gated recurrent unit to obtain a feature vector, and inputting the feature vector to preset fully connected layers to obtain a detection result; and optimizing the parameters of the recurrent neural network according to difference between the detection result and its corresponding distraction result label so as to obtain the distraction detection model, wherein the gated recurrent unit is used to control data flow direction and data flow amount in the recurrent neural network.

20. The computer-readable storage medium of claim 18, characterized in that, said preprocessing the EEG sample data to obtain preprocessed data comprises:
acquiring identification information of collection points corresponding to the EEG sample data, and determining first position information of electrodes corresponding to the identification information of the collection points on a data acquisition device;
determining, according to the first position information, second position information of emission sources on cerebral cortex corresponding to the collection points; and
removing artifacts in the EEG sample data according to the second position information, and slicing according to a preset slice period to obtain the preprocessed data, wherein the artifacts are EEG sample data corresponding to set positions to be removed.
